(19) United States
(12) Patent Application Publication
(10) Pub. No.: US 2025/0147525 A1
(43) Pub. Date: May 8, 2025

(54) MEDICAL DIAGNOSTIC APPARATUS, CONTROL METHOD FOR MEDICAL DIAGNOSTIC APPARATUS, AND CONTROL PROGRAM FOR MEDICAL DIAGNOSTIC APPARATUS

(71) Applicant: FUJIFILM Corporation, Tokyo (JP)

(72) Inventor: Taiga GOTO, Tokyo (JP)

(73) Assignee: FUJIFILM Corporation, Tokyo (JP)

(21) Appl. No.: 18/936,616

(22) Filed: Nov. 4, 2024

(30) Foreign Application Priority Data
Nov. 6, 2023 (JP) 2023-189592

(51) Int. Cl. G05D 3/20; A61B 6/04

(52) U.S. Cl. CPC G05D 3/20; A61B 6/0407

(57) ABSTRACT

A medical diagnostic apparatus includes a processor, and the processor is configured to: acquire a subject image which is an image of a subject placed on an examination table; acquire subject analysis information by analyzing the acquired subject image; and execute processing of controlling a height of the examination table based on the subject analysis information.

### CROSS-REFERENCE TO RELATED APPLICATION

This application claims priority from Japanese Patent Application No. 2023-189592, filed Nov. 6, 2023, the disclosure of which is incorporated herein by reference in its entirety.

### BACKGROUND

### Technical Field

The present disclosure relates to a medical diagnostic apparatus, a control method for the medical diagnostic apparatus, and a non-transitory storage medium storing a program. Related Art

In a medical diagnostic apparatus such as an X-ray CT apparatus and an MRI apparatus, a method of calculating a height at which it is easy to get on and off according to a height of a subject and changing a height of an examination table based on a calculation result is disclosed (JP2020-065920A, JP5191809B, and the like).

A height at which it is easy to get on and off an examination table varies depending on the physique, condition, age, and the like of a subject. However, in the related art, a technique of analyzing the state of the subject by using an image in which the subject is shown and controlling the height of the examination table of the medical diagnostic apparatus based on an analysis result has not been performed.

### SUMMARY

The present disclosure has been made in view of the above points, and an object of the present disclosure is to provide a medical diagnostic apparatus, a control method for the medical diagnostic apparatus, and a non-transitory storage medium storing a program that control a height of an examination table based on an analysis result of a subject using an image in which the subject is shown.

According to a first aspect of the present disclosure, there is provided a medical diagnostic apparatus comprising a processor, in which the processor is configured to: acquire a subject image which is an image of a subject placed on an examination table; acquire subject analysis information by analyzing the acquired subject image; and execute processing of controlling a height of the examination table based on the subject analysis information.

According to a second aspect of the present disclosure, in the medical diagnostic apparatus according to the first aspect, the subject image is a plurality of optical images obtained by imaging the subject from a plurality of directions, and the processor is configured to analyze the plurality of optical images to obtain the subject analysis information for each of the optical images, and control the height of the examination table based on a plurality of pieces of the subject analysis information.

According to a third aspect of the present disclosure, in the medical diagnostic apparatus according to the first or second aspect, the subject analysis information includes information on a physical feature of the subject.

According to a fourth aspect of the present disclosure, in the medical diagnostic apparatus according to any one of the first to third aspects, the subject analysis information includes information on a support member used in the subject for supporting the subject.

According to a fifth aspect of the present disclosure, in the medical diagnostic apparatus according to the fourth aspect, the processor is configured to, in a case where it is recognized that the subject is placed on a carrier different from the medical diagnostic apparatus, as the information regarding the support member, execute processing of controlling the height of the examination table to match a height of the carrier.

According to a sixth aspect of the present disclosure, in the medical diagnostic apparatus according to any one of the first to fifth aspects, the processor is configured to execute processing of controlling the height of the examination table at least in any of a case where the subject gets on the examination table or a case where the subject gets off the examination table.

According to a seventh aspect of the present disclosure, in the medical diagnostic apparatus according to the sixth aspect, the processor is configured to execute processing of controlling the height of the examination table by performing control related to an examination of the subject and discriminating between a case where the subject gets on the examination table and a case where the subject gets off the examination table based on progress information of the examination.

According to an eighth aspect of the present disclosure, in the medical diagnostic apparatus according to the first to seventh aspects, the processor has a readjustment mode allowing a readjustment of the height of the examination table after the height of the examination table is automatically controlled.

According to a ninth aspect of the present disclosure, in the medical diagnostic apparatus according to the eighth aspect, the processor is further configured to acquire an optical image of an operator who operates the height of the examination table, and in the readjustment mode, recognize a gesture of the operator from the acquired optical image and execute processing of readjusting the height of the examination table based on the gesture.

According to a tenth aspect of the present disclosure, in the medical diagnostic apparatus according to the eighth aspect, the processor is further configured to acquire a voice of an operator who operates the height of the examination table, and in the readjustment mode, recognize the acquired voice and execute processing of readjusting the height of the examination table based on the voice.

According to an eleventh aspect of the present disclosure, in the medical diagnostic apparatus according to the eighth aspect, the processor is configured to execute processing of readjusting the height of the examination table based on an operation of changing the height of the examination table in the readjustment mode.

According to a twelfth aspect of the present disclosure, in the medical diagnostic apparatus according to the eighth aspect, the processor is configured to execute processing of readjusting the height of the examination table by applying a preset offset value in the readjustment mode.

According to a thirteenth aspect of the present disclosure, in the medical diagnostic apparatus according to the first aspect, the processor is configured to execute processing of controlling the height of the examination table based on information during a past examination of the subject.

According to a fourteenth aspect of the present disclosure, in the medical diagnostic apparatus according to the thirteenth aspect, the processor is configured to obtain the subject analysis information by analyzing an optical image in which the subject is imaged and a past diagnostic image obtained in the past examination of the subject as the subject image, and execute processing of controlling the height of the examination table based on the subject analysis information obtained by analyzing the optical image and the past diagnostic image.

According to a fifteenth aspect of the present disclosure, in the medical diagnostic apparatus according to the thirteenth aspect, the processor is configured to execute processing of controlling the height of the examination table based on information on the height of the examination table during the past examination of the subject.

According to a sixteenth aspect of the present disclosure, there is provided a control method for a medical diagnostic apparatus, the control method comprising: via a processor, acquiring a subject image which is an image of a subject placed on an examination table;

acquiring subject analysis information by analyzing the acquired subject image; and executing processing of controlling a height of the examination table based on the subject analysis information.

According to a seventeenth aspect of the present disclosure, there is provided a control program for a medical diagnostic apparatus, the control program causing a computer to execute a process comprising: acquiring a subject image which is an image of a subject placed on an examination table; acquiring subject analysis information by analyzing the acquired subject image; and executing processing of controlling a height of the examination table based on the subject analysis information.

According to the present disclosure, it is possible to provide a medical diagnostic apparatus, a control method for the medical diagnostic apparatus, and a non-transitory storage medium storing a program that control a height of an examination table based on an analysis result of a subject using an image in which the subject is shown.

### BRIEF DESCRIPTION OF THE DRAWINGS

FIG. 1 is a diagram showing a schematic configuration of a medical diagnostic apparatus according to an embodiment of the disclosed technology.

FIG. 2 is a block diagram showing an example of a functional configuration of a console.

FIG. 3 is a diagram showing an example of past data of a subject.

FIG. 4 is a flowchart showing a flow of control of the medical diagnostic apparatus by the console.

FIG. 5 is a flowchart showing a flow of control of the medical diagnostic apparatus by the console.

### DESCRIPTION OF THE PREFERRED EMBODIMENTS

Hereinafter, an example of an embodiment of the present disclosure will be described with reference to the accompanying drawings. In each drawing, the same or equivalent components and parts are designated by the same reference numerals. Dimensional ratios in the drawings are exaggerated for convenience of description and may be different from actual ratios.

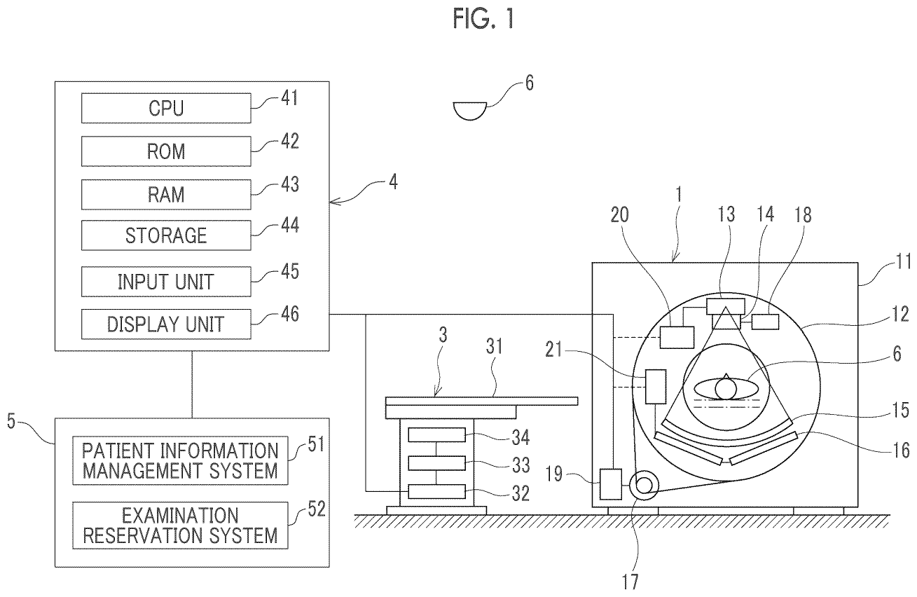

FIG. 1 is a diagram showing a schematic configuration of a medical diagnostic apparatus according to the present embodiment. In the present embodiment, a configuration of an embodiment of an X-ray CT apparatus as the medical diagnostic apparatus is shown. The medical diagnostic apparatus of the present disclosure is not limited to the X-ray CT apparatus, and can be applied to other medical diagnostic apparatuses as long as an examination table moves up and down.

The medical diagnostic apparatus according to the present embodiment includes a scanner 1, an examination table 3, and a console 4.

The scanner 1 is a portion that executes CT scanning. The scanner 1 comprises a gantry 11, a rotating plate 12 that has an opening at a center portion and is rotatably supported by the gantry 11, an X-ray tube device 13 fixed to the rotating plate 12, a collimator 14 provided in an X-ray emission port portion of the X-ray tube device 13, an X-ray detector 15 that is disposed to face the X-ray tube device 13 with the opening of the rotating plate 12 interposed therebetween, and a rotating plate drive unit 17 provided in the gantry 11. In addition, the rotating plate 12 of the scanner 1 comprises a collimator controller 18 that controls the collimator 14 to change an X-ray irradiation field, a rotating plate drive controller 19 that performs drive control of the rotating plate drive unit 17, an X-ray high-voltage generation device 20 that supplies power for generating X-rays to the X-ray tube device 13 and controls X-ray generation conditions, a data collection device 16 that collects the output of the X-ray detector 15, and a data transmission device 21 that transmits the data collected by the data collection device 16. The power and control signals are supplied to each unit provided in the rotating plate 12, and the data is taken out from each unit provided in the rotating plate 12, via a slip ring (not shown) provided between the gantry I and the rotating plate 12.

The examination table 3 moves a subject between an imaging preparation position and an imaging position. The subject is placed on a top plate 31. Although not shown, an up-down movement mechanism and a front-rear movement mechanism of the top plate 31 are provided. In addition, the examination table 3 is provided with an examination table controller 32, a top plate up-down movement controller 33, and a top plate front-rear movement controller 34 for controlling operations of the up-down movement mechanism and the front-rear movement mechanism of the top plate 31.

The console 4 controls the medical diagnostic apparatus that is the X-ray CT apparatus. The console 4 comprises a central processing unit (CPU) 41, a read only memory (ROM) 42, a random access memory (RAM) 43, and a storage 44. In addition, the console 4 may further comprise an input unit 45 and a display unit 46.

The CPU 41 which is an example of a processor is a central processing unit and executes various programs or controls each unit. That is, the CPU 41 reads out a program from the ROM 42 or the storage 44 and executes the program using the RAM 43 as a work area. The CPU 41 performs control of each configuration described above and various arithmetic processing according to the program recorded in the ROM 42 or the storage 44. In the present embodiment, a control program of the medical diagnostic apparatus that controls a height of the examination table 3 of the medical diagnostic apparatus is stored in the ROM 42 or the storage 44.

The ROM 42 stores various programs and various data. The RAM 43 as a work area temporarily stores the program or the data. The storage 44 is configured by a storage device such as a hard disk drive (HDD), a solid state drive (SSD), or a flash memory and stores various programs including an operating system and various data.

The input unit 45 includes a pointing device such as a mouse, and a keyboard, and is used to perform various inputs. The input unit 45 comprises an examination table operation unit that operates the height of the examination table 3.

The display unit 46 is, for example, a liquid crystal display, and displays various types of information. The display unit 46 may adopt a touch panel type to function as the input unit 45.

In the present embodiment, the console 4 is connected to a hospital information system 5 of a hospital in which the X-ray CT apparatus is installed. The hospital information system 5 comprises a patient information management system 51 and an examination reservation system 52.

The patient information management system 51 is a database in which personal information, medical care data, examination image data, examination data, medication data, and the like of a patient who has received medical care in the hospital in the past are stored. The personal information of the patient includes personal identification information such as a name, an ID, a date of birth, an age, and a gender, as well as physical information such as a height and a weight, and a past examination date. These pieces of personal information are input to the system in a case where the subject visits the hospital as a patient and receives medical care. The height and the weight of the patient are collected by the patient's self-reporting on a medical questionnaire or by actual measurement, and are input to the patient information management system 51. In addition, the medical care data, the examination image data, the examination data, and the medication data are input to the patient information management system each time the patient receives medical care, and an in-hospital patient database is created.

The examination reservation system 52 creates an examination schedule for that day for each system of the medical diagnostic apparatus equipped in the hospital based on information stored or written in the patient information management system, and distributes the created examination schedule to each system as data. The examination schedule includes an order of patients who undergo the examination on that day in the medical diagnostic apparatus and personal identification information and physical information of each patient. The content of the examination reservation system 52 is sequentially updated according to the progress of the medical care of that day, and the updated data is transmitted to each system each time the content is updated.

The console 4 uses an optical image captured by an imaging apparatus 6 in order to control the height of the top plate 31 of the examination table 3. The imaging apparatus 6 is installed in a room in which the medical diagnostic apparatus is installed, and is provided at a position where the imaging apparatus can image an appearance of a subject undergoing an examination. It is desirable that the position where the imaging apparatus 6 is installed is, for example, a position where there is no obstacle between the imaging apparatus 6 and the subject, such as a ceiling or a wall of a room or the gantry 11 of the scanner 1. In addition, the number of the imaging apparatuses 6 may be one, but a plurality of the imaging apparatuses 6 may be installed to image the appearance of the subject from different directions. Control processing of the examination table 3 using the optical image captured by the imaging apparatus 6 through the console 4 will be described in detail later.

In a case where the control program of the medical diagnostic apparatus is executed, the console 4 realizes various functions by using hardware resources described above. Functional configurations realized by the console 4 will be described.

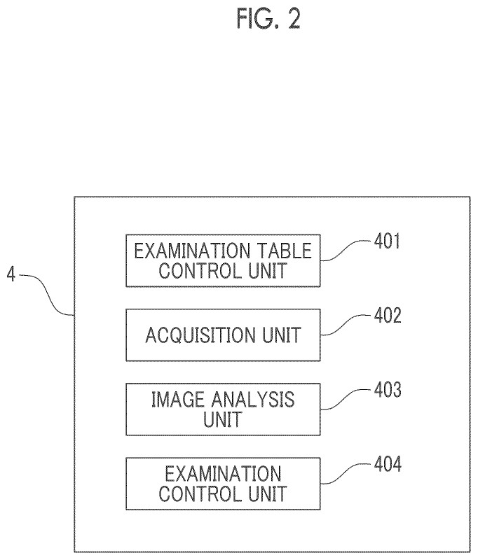

FIG. 2 is a block diagram showing an example of the functional configurations of the console 4.

As shown in FIG. 2, the console 4 has an examination table control unit 401, an acquisition unit 402, an image analysis unit 403, and an examination control unit 404, as the functional configurations. Each functional configuration is realized by the CPU 41 reading out and executing the control program of the medical diagnostic apparatus stored in the ROM 42 or the storage 44.

The examination table control unit 401 generates a control signal for controlling the height of the top plate 31 of the examination table 3. The control signal generated by the examination table control unit 401 is transmitted to the examination table controller 32 of the examination table 3. The examination table controller 32 changes the height of the top plate 31 based on the control signal. In a case where the subject is placed on the examination table 3, the examination table control unit 401 changes the height of the top plate 31 of the examination table 3 to an appropriate height for the examination with the scanner 1.

The acquisition unit 402 acquires a subject image which is an image in which the subject is captured. The subject image is an optical image obtained by imaging the subject with the imaging apparatus 6, but the subject image may be a past diagnostic image obtained in a past examination of the subject. The past diagnostic image may be an image obtained by a CT examination, an MRI examination, or the like. The acquisition unit 402 may acquire the past diagnostic image of the subject from, for example, the patient information management system 51.

The image analysis unit 403 analyzes the subject image acquired by the acquisition unit 402 to obtain subject analysis information. The image analysis unit 403 determines whether or not the subject is shown in the optical image captured by the imaging apparatus 6, and obtains the subject analysis information in a case where the subject is shown. The image analysis unit 403 may determine whether or not the subject is shown in the optical image captured by the imaging apparatus 6 based on, for example, whether or not a face of a person other than a hospital staff registered in advance is recognized, whether or not examination clothing is worn, or whether or not a person who has been identified by a technician is recognized. In addition, the image analysis unit 403 may recognize a person who is considered to be the subject by using a result of machine learning performed in advance.

The subject analysis information includes, for example, information on physical features, such as a height, a weight, a waist height, a leg length, and a physical condition. The image analysis unit 403 presumes the physical condition of the subject, for example, from a facial color, a gait (it is presumed that the physical condition is bad in a case where a walking stride is narrow), a posture (it is presumed that the physical condition is bad in a case where the waist is bent), and the like of the subject. In addition, the subject analysis information includes, for example, information on a support member used in the subject for supporting the subject. The support member is, for example, a splint, a crutch, a wheelchair, a stretcher, or the like. The image analysis unit 403 may obtain the subject analysis information from the subject image by using, for example, a trained model generated in advance by machine learning using the subject image. The obtaining of the subject analysis information from the subject image using the trained model refers to obtaining the subject analysis information from the output of the trained model by inputting the subject image to the trained model.

The examination table control unit 401 controls the height of the top plate 31 of the examination table 3 such that the subject easily gets on the top plate 31, by using the subject analysis information obtained by analyzing the subject image by the image analysis unit 403. The examination table control unit 401 controls the height of the top plate 31 based on, for example, a height, a waist height, and a leg length of the subject. In addition, the examination table control unit 401 may control the height of the top plate 31 to be lower than the height based on a height, a waist height, and a leg length of the subject, for example, in a case where the subject uses the support member. In addition, the examination table control unit 401 may control the height of the top plate 31 to be lower than the height based on a height, a waist height, and a leg length of the subject, for example, in a case where the weight of the subject is heavier than a specified ratio to a standard value. In addition, the examination table control unit 401 may control the height of the top plate 31 to be lower than the height based on a height, a waist height, and a leg length of the subject, for example, in a case where the age of the subject is equal to or greater than a predetermined value. In addition, the examination table control unit 401 may control the height of the top plate 31 to be lower than the height based on a height, a waist height, and a leg length of the subject, for example, in a case where the physical condition of the subject is bad.

The examination table control unit 401 can control the height of the top plate 31 of the examination table 3 by using the subject analysis information, so that the medical diagnostic apparatus can set the height of the top plate 31 to a height at which the subject can easily get on.

In a case where a plurality of the imaging apparatuses 6 are installed, the image analysis unit 403 may obtain the subject analysis information by using a plurality of subject images obtained by imaging with each of the imaging apparatuses 6. In this case, the examination table control unit 401 may control the height of the top plate 31 of the examination table 3 based on the certainty of the subject analysis information obtained by analyzing each subject image. For example, in a case where the subject analysis information obtained by analyzing the subject image captured by the imaging apparatus 6 installed on the ceiling by the image analysis unit 403 is compared with the subject analysis information obtained by analyzing the subject image captured by the imaging apparatus 6 installed on the wall by the image analysis unit 403, and the certainty of the subject analysis information obtained from the subject image captured by the imaging apparatus 6 installed on the wall is higher, the examination table control unit 401 may control the height of the top plate 31 of the examination table 3 by using the subject analysis information obtained from the subject image captured by the imaging apparatus 6 installed on the wall. Even in a case where the subject analysis information is obtained from the subject image captured by the same imaging apparatus 6, a value of the certainty may vary depending on a situation of the subject. For example, the value of the certainty of the subject analysis information may be different between a case where the subject is in a standing position, a case where the subject is in a sitting position, and a case where the subject is in a recumbent position. Therefore, the image analysis unit 403 can control the height of the top plate 31 of the examination table 3 appropriately according to the situation of the subject by controlling the height of the top plate 31 of the examination table 3 based on the value of the certainty of the subject analysis information.

In a case where the subject analysis information indicates that the subject is riding on a wheelchair as a result of analyzing the subject image by the image analysis unit 403, the examination table control unit 401 may control the height of the top plate 31 of the examination table 3 by using the subject analysis information obtained from the subject image captured by the imaging apparatus 6 installed on the wall. In a case where the subject is riding on a wheelchair, it is difficult to recognize a leg length and a waist height of the subject in the optical image captured by the imaging apparatus 6 installed on the ceiling. Therefore, the image analysis unit 403 may obtain the subject analysis information by using the subject image captured by the imaging apparatus 6 installed at a position that allows imaging from a side surface such as the wall and the gantry 11, in a case where the subject is riding on the wheelchair.

In a case where the subject analysis information indicates that the subject is placed on a carrier (for example, a stretcher) different from the medical diagnostic apparatus as a result of analyzing the subject image by the image analysis unit 403, the examination table control unit 401 may perform control such that the height of the top plate 31 of the examination table 3 matches a height of the carrier.

The examination control unit 404 controls the examination of the subject by the medical diagnostic apparatus. In a case where the examination control unit 404 determines to start a predetermined examination based on progress information of the examination in the medical diagnostic apparatus, the examination control unit 404 moves the examination table 3 into the rotating plate 12. In addition, in a case where the examination control unit 404 determines that the predetermined examination is terminated based on the progress information of the examination in the medical diagnostic apparatus, the examination control unit 404 moves the examination table 3 to the outside of the rotating plate 12.

The examination table control unit 401 discriminates a timing at which the examination is terminated, that is, a timing at which the examination table 3 is moved to the outside of the rotating plate 12 and the subject gets off the examination table, and controls the height of the top plate 31 of the examination table 3 such that the subject can easily get off the examination table 3. Whether or not the examination has been terminated may be determined by the examination control unit 404 based on the progress information of the examination, may be determined by the image analysis unit 403 from the optical image captured by the imaging apparatus 6 showing that the subject has come out of the rotating plate 12 in a state in which the subject is lying on the examination table, or may be determined by the examination table control unit 401 detecting a weight of an object placed on the examination table 3. The examination table control unit 401 detects the weight of the object placed on the examination table 3, and determines that a person is placed on the examination table 3 in a case where the weight of the object placed on the examination table 3 is about a body weight of a person. In a case where the weight of the object placed on the examination table 3 is about a body weight of a human body (for example, 50 to 80 kilograms) and the examination table 3 comes out of the rotating plate 12, the examination control unit 404 determines that the examination of the subject is terminated. In this case, the examination table control unit 401 controls the height of the top plate 31 of the examination table 3. On the other hand, in a case where the examination table control unit 401 detects that the weight of the object on the examination table 3 is light, the examination control unit 404 determines that the examination of the subject has not yet started. In that case, the examination table control unit 401 controls the height of the top plate 31 of the examination table 3 such that the subject can easily get on the examination table 3. In addition, depending on the content of the examination using the medical diagnostic apparatus, there may be a case where the examination table 3 is temporarily removed from the gantry 11 in the middle of the examination and the subject gets off the examination table 3. In that case, the examination table control unit 401 may control the height of the top plate 31 of the examination table 3 at a point in time at which the examination table 3 is moved to the outside of the rotating plate 12, not at the termination of the examination, but even in the middle of the examination.

Even in a case where the examination table control unit 401 controls the height of the top plate 31 of the examination table 3 to match the subject by using the subject analysis information, there is a possibility that the height does not match the subject in practice. In this regard, the examination table control unit 401 may have a readjustment mode allowing a readjustment of the height of the top plate 31 of the examination table 3 after the height of the top plate 31 of the examination table 3 is automatically controlled.

The examination table control unit 401 enters a readjustment mode in a case where a predetermined operation of the operator is detected as a trigger. Examples of the detection of the predetermined operation include detection of a predetermined gesture of the operator from the optical image captured by the imaging apparatus 6, detection of a predetermined voice command of the operator by a microphone installed at a predetermined location, and detection of an operation in which the operator operates a height operation unit that controls the height of the examination table 3. The microphone may be installed independently or integrally with the imaging apparatus 6. The acquisition unit 402 may acquire the voice command. In addition, for example, the height operation unit that controls the height of the examination table 3 may be provided as the input unit 45, or may be a foot switch, a hand switch, a switch provided next to the examination table 3, a virtual switch projected on a wall surface, or the like.

In a case where the readjustment mode is entered, the examination table control unit 401 readjusts the height of the top plate 31 of the examination table 3 based on a predetermined operation of the operator. The predetermined operation is, for example, a predetermined gesture operation of the operator. The gesture may be, for example, a gesture indicating an upward or downward direction with a finger, and the examination table control unit 401 moves the top plate 31 of the examination table 3 in the direction of the gesture during the continuation of the gesture. In addition, the predetermined operation is, for example, a predetermined voice command of the operator. The voice command may be “x centimeters up”, or “x centimeters down”, or may be “a little more up”, or “a little more down”, or the like. The examination table control unit 401 moves the top plate 31 of the examination table 3 based on the voice command. In addition, the predetermined operation is, for example, an operation of a height operation unit by the operator. The examination table control unit 401 moves the top plate 31 of the examination table 3 in response to the operation of the height operation unit. In addition, in a case where the readjustment mode is entered, the examination table control unit 401 may change the height of the top plate 31 of the examination table 3 by applying a predetermined offset value. In addition, in a case where a voice command that is not specific, such as “a little more up” or “a little more down”, is input, the examination table control unit 401 may change the height of the top plate 31 of the examination table 3 by the predetermined offset value.

The examination table control unit 401 may control the height of the top plate 31 of the examination table 3 based on past data of the subject. For example, the past data of the subject is stored in the patient information management system 51 and is acquired by the acquisition unit 402.

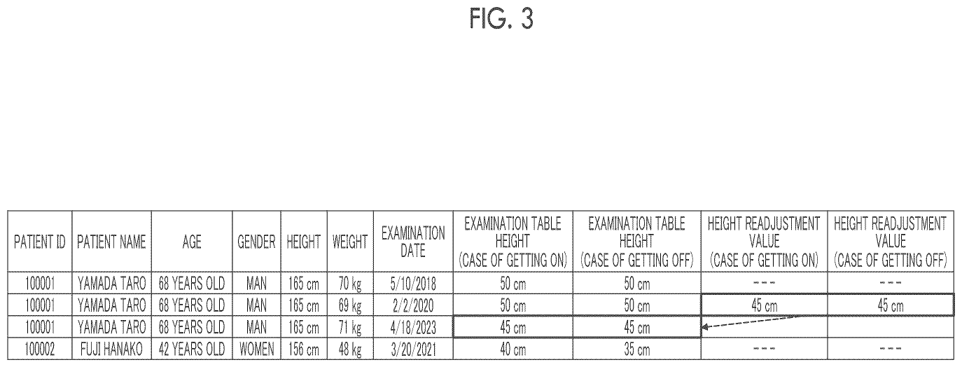

FIG. 3 is a diagram showing an example of the past data of the subject. As shown in FIG. 3, the past data of the subject includes a patient ID for identifying the subject, a name, a gender, a height, a weight, and an examination date. The values of the height and the weight may be values based on self-reporting of the subject, actually measured values, or values in the subject analysis information. In addition, as shown in FIG. 3, in the past data of the subject, for each past examination of the subject, a value of a height in a case where the subject gets on the examination table 3, a value of a height in a case where the subject gets off the examination table 3, and values of heights in a case where the subject gets on the examination table 3 and gets off the examination table 3 after being readjusted by the readjustment mode are recorded. The examination table control unit 401 may specify a target subject from the image captured by the imaging apparatus 6 or the information recorded in the examination reservation system 52, and may control the height of the top plate 31 of the examination table 3 based on data of the subject in a case where the data of the subject exists in the past data of the subject. In a case where a value of the height of the examination table 3 after being readjusted by the readjustment mode in the most recent examination is recorded, the examination table control unit 401 may set the height of the top plate 31 of the examination table 3 to the value of the height after being readjusted by the readjustment mode.

In addition, the examination table control unit 401 may control the height of the top plate 31 of the examination table 3 based on the past diagnostic image obtained in the past examination of the subject as the past data of the subject. In this case, the image analysis unit 403 may analyze the past diagnostic image of the subject to obtain the subject analysis information, and the examination table control unit 401 may control the height of the top plate 31 of the examination table 3 based on the subject analysis information. Here, the subject analysis information obtained by analyzing the past diagnostic image may include positions of lower limbs and a pelvis of the subject.

In the above description, a case where the examination table control unit 401 is included in the console 4 has been described, but the present disclosure is not limited to this example. The function of the examination table control unit 401 may be included in a processor of a board built in the gantry 11 by reading out and executing the control program of the medical diagnostic apparatus by the processor of the board built in the gantry 11. The processor reads out and executes the control program of the medical diagnostic apparatus to control the height of the top plate 31 of the examination table 3 by transmitting and receiving information to and from the console 4.

Next, an operation of the console 4 will be described.

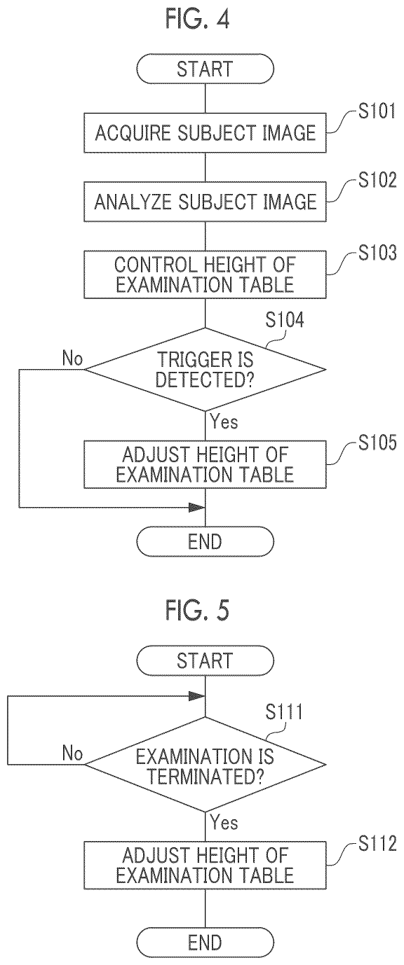

FIG. 4 is a flowchart showing a flow of control of the medical diagnostic apparatus by the console 4. The CPU 41 reads out the control program of the medical diagnostic apparatus from the ROM 42 or the storage 44, and loads and executes the control program in the RAM 43, thereby performing control processing of the medical diagnostic apparatus. The flowchart shown in FIG. 4 is executed in a case where the subject is examined by the medical diagnostic apparatus, that is, before the subject gets on the examination table 3.

In step S101, the CPU 41 acquires the optical image captured by the imaging apparatus 6 as the subject image.

After step S101, in step S102, the CPU 41 analyzes the subject image to obtain the subject analysis information.

After step S102, in step S103, the CPU 41 controls the height of the top plate 31 of the examination table 3 such that the subject easily gets on the examination table 3 based on the subject analysis information.

After step S103, in step S104, the CPU 41 determines whether or not a predetermined trigger is detected. In a case where the predetermined trigger is detected (step $104; Yes), in step S105, the CPU 41 enters a height readjustment mode, and adjusts the height of the top plate 31 of the examination table 3 based on the operation from the operator. On the other hand, in a case where the predetermined trigger is not detected (step S104; No), the CPU 41 terminates a series of processing.

FIG. 5 is a flowchart showing a flow of control of the medical diagnostic apparatus by the console 4. The CPU 41 reads out the control program of the medical diagnostic apparatus from the ROM 42 or the storage 44, and loads and executes the control program in the RAM 43, thereby performing control processing of the medical diagnostic apparatus. The flowchart shown in FIG. 5 is executed while the examination in the medical diagnostic apparatus is executed.

In step S111, the CPU 41 waits until the examination in the medical diagnostic apparatus is terminated. In a case where the examination in the medical diagnostic apparatus is terminated (step S111; Yes), in step S112, the CPU 41 controls the height of the top plate 31 of the examination table 3 such that the subject easily gets off the examination table 3, based on the subject analysis information.

Although the embodiment of the present disclosure has been described in detail with reference to the accompanying drawings, the technical scope of the present disclosure is not limited to such an example. It is apparent that those having ordinary knowledge in the technical field of the present disclosure can conceive various change examples or modification examples within the technical idea described in the claims, and it is also understood that these change examples or modification examples belong to the technical scope of the present disclosure.

In addition, the effects described in the above embodiment are merely explanatory or exemplary, and the present disclosure is not limited to the effects described in the above embodiment. That is, the technology according to the present disclosure can achieve other effects that are obvious to those having ordinary knowledge in the technical field of the present disclosure in addition to the effects described in the above embodiment, or instead of the effects described in the above embodiment.

The control processing of the medical diagnostic apparatus that is executed by causing the CPU to read software (program) in each embodiment may be executed by various processors other than the CPU. In this case, examples of the processor include a programmable logic device (PLD) of which a circuit configuration may be changed after manufacturing, such as a field-programmable gate array (FPGA), and a dedicated electric circuit which is a processor having a circuit configuration specially designed for executing specific processing, such as an application specific integrated circuit (ASIC). Further, the control processing of the medical diagnostic apparatus may be executed by one of the various processors, or executed by the combination of the same or different kinds of two or more processors (for example, a combination of a plurality of FPGAs, a combination of the CPU and the FPGA, or the like). In addition, a hardware structure of the various processors is more specifically an electric circuit in which circuit elements such as semiconductor elements are combined.

In addition, in the above embodiment, an aspect has been described in which the control program of the medical diagnostic apparatus is stored (installed) in the ROM or the storage in advance, but the present disclosure is not limited to this. The program may be provided in a form of being recorded on a non-transitory recording medium such as a compact disk read only memory (CD-ROM), a digital versatile disk read only memory (DVD-ROM), and a universal serial bus (USB) memory. Further, the program may be downloaded from an external device via a network.

What is claimed is:

1. A medical diagnostic apparatus comprising a processor,
wherein the processor is configured to:
acquire a subject image which is an image of a subject placed on an examination table;
acquire subject analysis information by analyzing the acquired subject image; and
execute processing of controlling a height of the examination table based on the subject analysis information.

2. The medical diagnostic apparatus according to claim 1,
wherein the subject image is a plurality of optical images obtained by imaging the subject from a plurality of directions, and
the processor is configured to analyze the plurality of optical images to obtain the subject analysis information for each of the optical images, and execute processing of controlling the height of the examination table based on a plurality of pieces of the subject analysis information.

3. The medical diagnostic apparatus according to claim 1,
wherein the subject analysis information includes information on a physical feature of the subject.

4. The medical diagnostic apparatus according to claim 1,
wherein the subject analysis information includes information on a support member used in the subject for supporting the subject.

5. The medical diagnostic apparatus according to claim 4,
wherein the processor is configured to, in a case where it is recognized that the subject is placed on a carrier different from the medical diagnostic apparatus, as the information regarding the support member, execute processing of controlling the height of the examination table to match a height of the carrier.

6. The medical diagnostic apparatus according to claim 1,
wherein the processor is configured to execute processing of controlling the height of the examination table at least in any of a case where the subject gets on the examination table or a case where the subject gets off the examination table.

7. The medical diagnostic apparatus according to claim 6,
wherein the processor is configured to execute processing of controlling the height of the examination table by performing control related to an examination of the subject and discriminating between a case where the subject gets on the examination table and a case where the subject gets off the examination table based on progress information of the examination.

8. The medical diagnostic apparatus according to claim 1,
wherein the processor has a readjustment mode allowing a readjustment of the height of the examination table after the height of the examination table is automatically controlled.

9. The medical diagnostic apparatus according to claim 8,
wherein the processor is further configured to acquire an optical image of an operator who operates the height of the examination table, and in the readjustment mode, recognize a gesture of the operator from the acquired optical image and execute processing of readjusting the height of the examination table based on the gesture.

10. The medical diagnostic apparatus according to claim 8,
wherein the processor is further configured to acquire a voice of an operator who operates the height of the examination table, and in the readjustment mode, recognize the acquired voice and execute processing of readjusting the height of the examination table based on the voice.

11. The medical diagnostic apparatus according to claim 8,
wherein the processor is configured to execute processing of readjusting the height of the examination table based on an operation of changing the height of the examination table in the readjustment mode.

12. The medical diagnostic apparatus according to claim 8,
wherein the processor is configured to execute processing of readjusting the height of the examination table by applying a preset offset value in the readjustment mode.

13. The medical diagnostic apparatus according to claim 1,
wherein the processor is configured to execute processing of controlling the height of the examination table based on information during a past examination of the subject.

14. The medical diagnostic apparatus according to claim 13,
wherein the processor is configured to obtain the subject analysis information by analyzing an optical image in which the subject is imaged and a past diagnostic image obtained in the past examination of the subject as the subject image, and execute processing of controlling the height of the examination table based on the subject analysis information obtained by analyzing the optical image and the past diagnostic image.

15. The medical diagnostic apparatus according to claim 13,
wherein the processor is configured to execute processing of controlling the height of the examination table based on information on the height of the examination table during the past examination of the subject.

16. A control method for a medical diagnostic apparatus, the control method comprising:
via a processor,
acquiring a subject image which is an image of a subject placed on an examination table;
acquiring subject analysis information by analyzing the acquired subject image; and
executing processing of controlling a height of the examination table based on the subject analysis information.

17. A non-transitory storage medium storing a program, the program causing a computer to execute a process comprising:
acquiring a subject image which is an image of a subject placed on an examination table;
acquiring subject analysis information by analyzing the acquired subject image; and
executing processing of controlling a height of the examination table based on the subject analysis information.
